(19) United States
(12) Patent Application Publication
(10) Pub. No.: US 2023/0178197 A1
(43) Pub. Date: Jun. 8, 2023

(54) TECHNIQUES FOR PREDICTING IMMUNOSUPPRESSION STATUS

(71) Applicant: nference, inc., Cambridge, MA (US)

(72) Inventors: Travis K. HUGHES, Cambridge, MA (US); Aiveliagaram J. VENKATAKRISHNAN, Cambridge, MA (US); Patrick J. LENEHAN, Cambridge, MA (US); Venkataramanan SOUNDARARAJAN, Andover, MA (US); Murali ARAVAMUDAN, Andover, MA (US)

(21) Appl. No.: 18/074,283

(22) Filed: Dec. 2, 2022

Related U.S. Application Data
(60) Provisional application No. 63/285,880, filed on Dec. 3, 2021.

(51) Int. Cl. G16H 10/60; G16H 50/30

(52) U.S. Cl. CPC G16H 10/60; G16H 50/30

(57) ABSTRACT

Techniques for predicting the immunosuppression status of an individual patient in a computing environment are disclosed. In one particular embodiment, the techniques may be realized as a method comprising receiving a set of medical records associated with a patient, extracting a set of immunosuppression features based on the set of medical records, estimating, a likelihood of immunosuppression of the patient based on the set of immunosuppression features, generating an immunosuppression output comprising one or more features among the set of immunosuppression features and an immunosuppression classification based on the likelihood of immunosuppression of the patient, and displaying the immunosuppression output through at least one interface.

### RELATED APPLICATIONS

This application claims priority to U.S. Provisional Application No. 63/285,880, entitled “Techniques for Predicting Immunosuppression Status,” filed Dec. 3, 2021, which is incorporated by reference herein in its entirety.

### FIELD OF THE DISCLOSURE

The present disclosure relates to identifying immunosuppressed patients and generating point of care outputs for individual patients.

### BACKGROUND OF THE DISCLOSURE

Clinicians, doctors, and health professionals must properly diagnose patients to provide effective treatment options. Among the patient population, immunosuppressed patients in particular represent a higher risk for negative healthcare outcomes. For example, a patient who is brought into a hospital may acquire an infection more easily due to the patient's own weakened immune system. Therefore, health professionals must quickly and accurately identify immunocompromised patients so that they may provide the proper treatment and use the proper protocols to reduce the risk of a negative outcome.

A clinician traditionally identifies immunosuppressed patients through manual chart review. For example, a doctor may examine a particular patient's history of medication, drugs, past surgical procedures, known diseases, or other factors to determine whether a patient has a weakened immune system. However, such a traditional approach presents issues in terms of accuracy and practicality. Because different doctors may consider different factors when evaluating immunosuppression data, it is difficult to standardize the immunosuppression analysis to arrive at a predictable outcome. Furthermore, such an analysis requires detailed and specialized knowledge of a patient's particular medical history, which often requires lengthy manual review of the patient's records. In some instances, the patient may bear the burden of seeking particular treatments based on his own immunosuppression status, but the patient will typically not have the necessary knowledge to do so without the specialized knowledge of a health professional. Therefore, there is a need for systems and methods to provide health professionals and patients with an accurate prediction of a patient's immunosuppression status using patient records and medical histories based on features of immunosuppression identified in large clinical data sets.

### SUMMARY OF THE DISCLOSURE

Techniques for predicting the immunosuppression status of an individual patient in a computing environment are disclosed. In one particular embodiment, the techniques may be realized as a method comprising receiving a set of medical records associated with a patient, extracting a set of immunosuppression features based on the set of medical records, estimating, a likelihood of immunosuppression of the patient based on the set of immunosuppression features, generating an immunosuppression output comprising one or more features among the set of immunosuppression features and an immunosuppression classification based on the likelihood of immunosuppression of the patient, and displaying the immunosuppression output through at least one interface.

In another particular embodiment, the techniques may be realized as a system for predicting the immunosuppression status of an individual patient comprising at least one computer processor communicatively coupled to and configured to operate in the system, wherein the at least one computer processor is further configured to perform the steps in the above-described method.

In another particular embodiment, the techniques may be realized as an article of manufacture for predicting the immunosuppression status of an individual patient comprising a non-transitory processor readable medium and instructions stored on the medium, wherein the instructions are configured to be readable from the medium by at least one computer processor and to thereby cause the at least one computer processor to operate so as to perform the steps in the above-described method.

### BRIEF DESCRIPTION OF THE DRAWINGS

Various objectives, features, and advantages of the disclosed subject matter can be more fully appreciated with reference to the following detailed description of the enclosed subject matter when considered in connection with the following drawings, in which like reference numerals identify like elements. The following drawings should not be construed as limiting the present disclosure and are intended to be illustrative only.

FIG. 1 is a diagram illustrating an example of an immunocompromised prediction system, according to an embodiment of the present disclosure.

FIG. 2 is a diagram illustrating example factors to identify immunosuppression status, according to an embodiment of the present disclosure.

FIG. 3 is a diagram illustrating example validation data used to identify immunosuppression status, according to an embodiment of the present disclosure.

FIG. 4 is a diagram illustrating an example patient timeline, according to an embodiment of the present disclosure.

FIG. 5 is a diagram illustrating an example individualized patient result from the immunocompromised prediction system, according to an embodiment of the present disclosure.

FIG. 6 is a diagram illustrating an example workflow for preparing and validating data for the immunocompromised prediction system, according to an embodiment of the present disclosure.

FIG. 7 is a diagram illustrating an example flow diagram to return and display an individualized point of care output, according to an embodiment of the present disclosure.

FIG. 8 is a diagram illustrating an example computing system upon which an embodiment of the invention may be implemented.

### DETAILED DESCRIPTION OF EMBODIMENTS

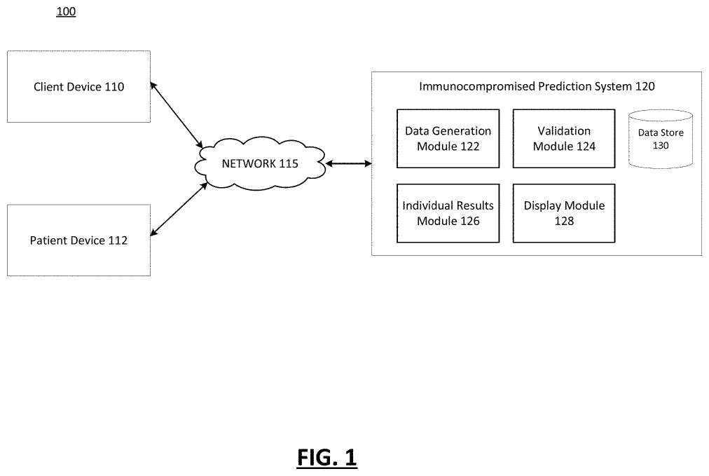

FIG. 1 illustrates an example of an immunocompromised prediction environment. In some embodiments, the immunocompromised prediction environment comprises a client device 110, a patient device 112, and an immunocompromised prediction system 120 connected through a network 115. In some embodiments, immunocompromised prediction system 120 may comprise a data generation module 122, a validation module 124, an individual results module 126, a display module 128, and a data store 130. Data generation module 122, may collect and compile patient record data to generate a curated set of training data for a machine learning model. In some embodiments, the features collected and considered by the immunocompromised prediction system 120 may be obtained from structured data commonly utilized in electronic medical records systems. For example, data generation module 122 may obtain structured data referring to disease codes (e.g., ICD-10, ICD-9, SNOMED, etc.), procedure codes (e.g., CPT), and medication records (e.g., documentation of medications ordered or administered to patients). In some embodiments, data generation module 122 may extract specific information from each patient that may be relevant to predicting immunosuppression status. For example, data generation module 122 may obtain immunodeficiency diagnosis codes, cancer diagnosis codes, opportunistic infection codes, autoimmune disease diagnosis codes, chronic disease diagnosis codes, a list of immunosuppressive medications ordered and administered, and transplant procedure codes for each patient. The data generation module 122 may further filter such data by limiting the collection to a specific time frame (e.g., immunosuppressive medications ordered or administered 1 year before the current date). The data generation module 122 may also perform additional operations over the entire data set. For example, module 122 may calculate the number of unique entries in the data set in a given time period (e.g., the total number of unique chronic disease diagnosis codes). The immunocompromised prediction system 120 may therefore represent each patient as a vector of collected immunosuppression features. In some embodiments, module 122 may capture additional clinical features not necessarily represented in structured clinical data. For example, the prediction system 120 may use natural language processing models (e.g., BERT/bidirectional encoder representations from transformers) to extract unstructured notes annotated by a clinician in the electronic health records. In some embodiments, module 122 may identify instances where a clinician has documented that a patient is immunosuppressed by detecting if the clinician included notes classifying the particular patient as immunosuppressed. In some embodiments, the data collected by module 122 may be stored in data store 130.

The validation module 124 may evaluate the predictive power of each feature collected by data generation module 122. The validation module 124 may do so by first separating the patients into one or more cohorts of patients. For example, validation module 124 may generate a group of patients where each patient is associated with clinician records that mention immunosuppression (e.g., the Immunosuppressed Cohort). The validation module 124 may also group patients who are not associated with immunosuppression clinician records in a separate group (e.g., the Immunocompetent Cohort). In some embodiments, the validation module 124 may group patients along other factors, such as by the patient's age or sex, and each cohort may be associated with a target index date, such as the first date on which a clinical note indicated that a particular patient was immunosuppressed. In some embodiments, validation module 124 may generate a contingency table and generate a numerical score for each individual feature (e.g., a crude odds ratio, regression coefficient, or Fisher exact p-value). In some embodiments, a feature may be considered relevant to predicting immunosuppression status if the numerical score approaches a certain threshold. For example, if the presence of an autoimmune disease is associated with an odds ratio greater than 1 or a p-value less than 0.05. The validation module 124 may generate a binary field for each patient that tracks the presence of features significant to detect immunosuppression status. For example, if the validation module 124 determined that none of a patient's medical features is associated with an odds ratio greater than 1, then that specific patient is associated with a binary field with the numerical value 0 representing that the patient has no features that would cause a risk of immunosuppression. In contrast, if even one of the patient's medical features is associated with an odds ratio greater than 1 or a p-value greater than 0.05, then the patient is associated with a binary field with the numerical value 1. In some embodiments, the validation module 124 may perform a receiver operating characteristic (“ROC”) analysis to determine the sensitivity and specificity for immunosuppression classification. The validation module 124 may perform a logistic regression analysis to assess the association of each feature to an actual immunosuppression status. In doing so, the validation module 124 may calculate specific regression coefficients and a confidence interval for each immunosuppression feature. In some embodiments, the validation module 124 may also perform an ROC analysis to determine the sensitivity and specificity of immunosuppression classification.

While validation module 124 may perform calculations for a whole array of patients, in some embodiments system 120 may include an individual results module 126 that outputs individualized results for specific patients. The results returned by individual results module 126 may be displayed through a graphical user interface via display module 128.

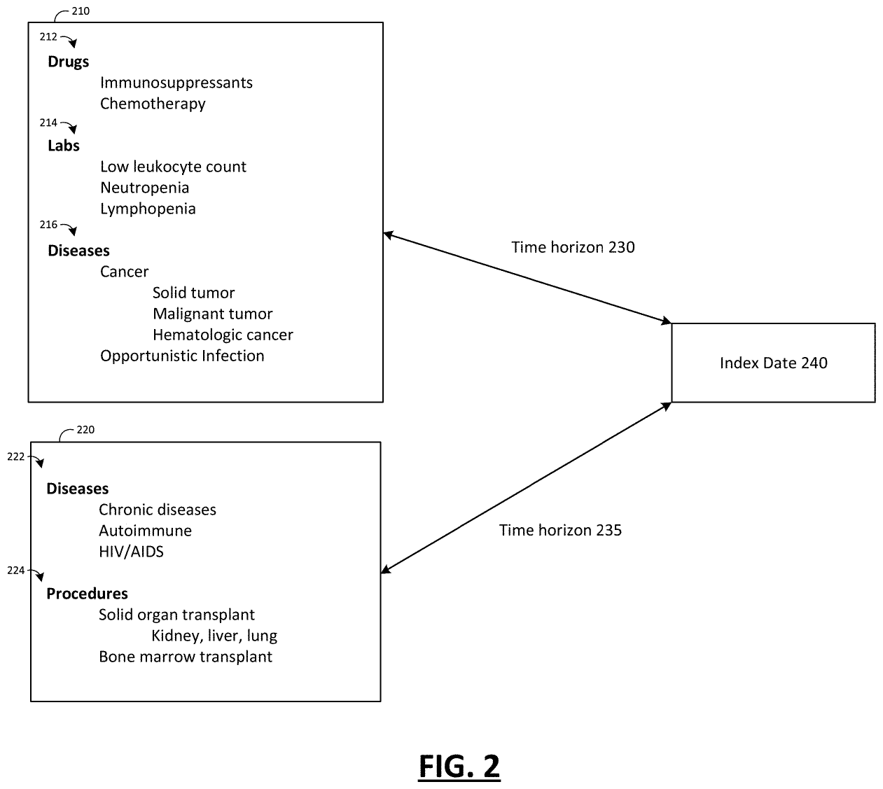

FIG. 2 illustrates an example of immunosuppression features considered by the prediction system 120 described above in FIG. 1. The prediction system 120 may evaluate a number of factors based at least partly on a set time horizon before a specific index date 240. For example, window 210 may display a list of features from a patient's records from a time horizon 230 before the index date 240 (e.g., 1 year before the current date). In this example, window 210 may include categories 212, 214, and 216 which give a picture of a patient's particular medical history during the time horizon 230. Window 210 may further list detailed information relevant to each of the categories 212, 214, 216. For example, window 210 lists the particular drugs associated with the patient (e.g., immunosuppressants, chemotherapy), lab results (e.g., low leukocyte count, neutropenia, lymphopenia), and diagnosed diseases (e.g., cancer or opportunistic infections). Window 220 may provide a similar list of the patient's medical history but may focus on a different time horizon 235. For example, window 220 may provide a list of any chronic or long-term diseases 222 or significant surgical procedures 224 that occurred any time before the index date 240. Although FIG. 2 only lists two windows with two different time horizons, it will be appreciated that any number of windows, time horizons, and features may be considered in other embodiments.

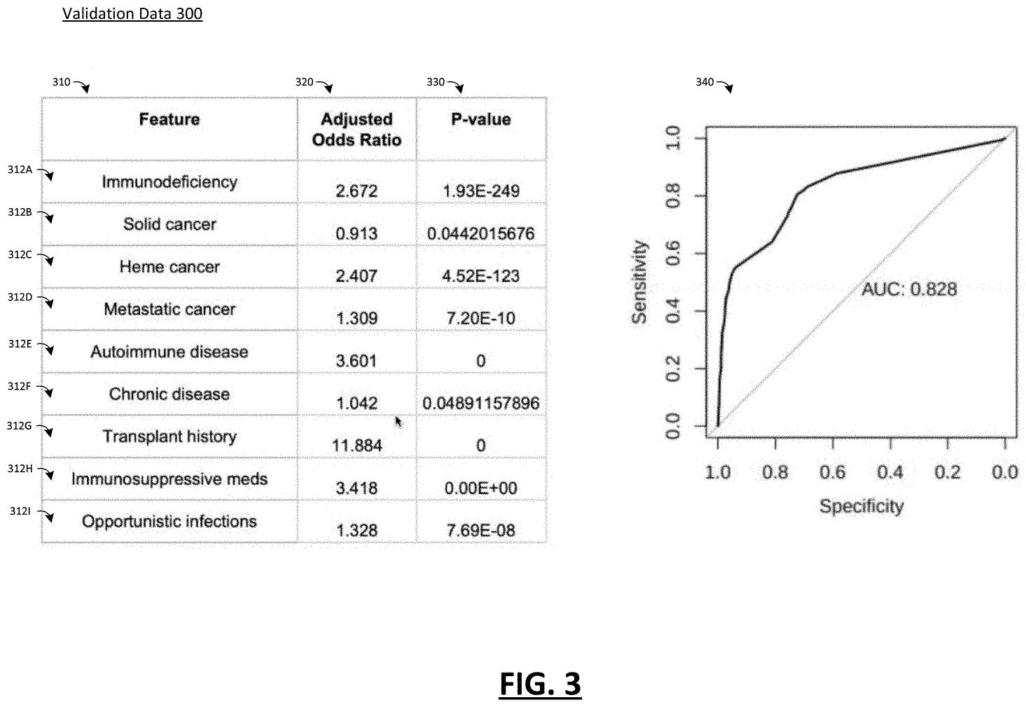

FIG. 3 illustrates data that may be validated and results that may be output by validation module 124. Validation data 300 comprises a feature column 310, a ratio column 320, and p-value column 330 and graph 340. Column 310 provides a list of features 312A through 312I that each represent an immunosuppression feature examined by the prediction system 120. The features listed in column 310 may be extracted from structured data from electronic health records, unstructured data such as notes annotated by a clinician, or a combination of both structured and unstructured data fields. As mentioned above in connection with FIG. 1, validation module 124 may compare the prevalence of immunosuppression features between cohorts by generating numerical scores for each immunosuppression feature listed in column 310. In some embodiments, the prediction system 120 may calculate the total number of instances where an immunosuppression feature is absent or present in a control group of immunocompetent patients versus the total number of instances where an immunosuppression feature is absent or present in the immunosuppressed cohort. In the example of FIG. 3, column 320 lists the calculated adjusted odds ratio while column 330 lists the calculated p-value for each of the immunosuppression features 312A through 312I. In some embodiments, a particular immunosuppression feature may be considered relevant to determining immunosuppression status if the odds ratio listed in column 320 passes the numeric threshold of 1 or if the p-value listed in column 330 is below the numerical threshold of 0.05. In some embodiments, the validation module 124 may perform an ROC analysis to determine the sensitivity and specificity for immunosuppression classification, which may be represented in graph 340. Graph 340 may also include a calculation of the area under the curve for the sensitivity versus specificity curve.

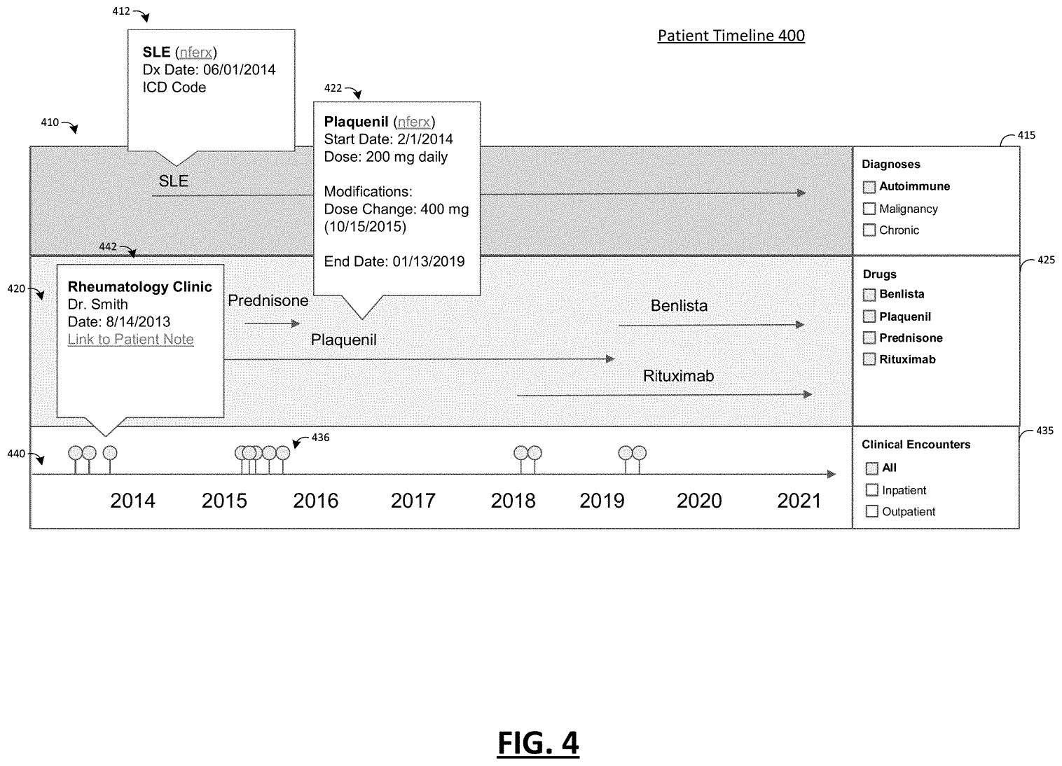

FIG. 4 illustrates an example patient timeline. Patient timeline 400 visually depicts a number of immunosuppression features relevant for predicting a patient's immunosuppression status. The patient timeline 400 may comprise a diagnoses timeline 410, a drugs timeline 420, and a clinical encounters timeline 440. Diagnoses timeline 410 may list any condition or disease diagnosed by a clinician over a specific time period (e.g., the past 7 years). The conditions listed in diagnoses timeline 410 may be positioned such that it is visually apparent when the specific diagnosis occurred. For example, FIG. 4 depicts a systemic lupus erythematosus (SLE) diagnosis dated in the year 2014. Patient timeline 400 may also allow a clinician or other user to view more detailed information about a particular diagnosis in the diagnoses timeline 410. For example, a user may hover a field with a computer mouse driven pointer, click, or touch the SLE diagnosis to display window 412, which displays further information such as the exact date of the diagnosis (e.g., Jun. 1, 2014) and ICD code. Furthermore, diagnoses timeline 410 may be accompanied by diagnoses filter 415, which may allow a user to actively select specific diagnoses to display in diagnoses timeline 410. Drugs timeline 420 displays drugs administered or used and the timing of when they were first administered and used. The user may filter out or view specific drugs using drugs filter 425, and the user may view additional details related to the drugs in window 422. For example, window 422 displays the first date when the selected drug Plaquenil was administered, the initial prescribed dose, modifications to the dosage, and the end date for using the drug. Clinical encounters timeline 440 provides a visual depiction of each clinic visit, hospital visit, or other encounter with health professionals over a specific time period, such as with pin 436. Like with timelines 410 and 420, a user may adjust filter 435 to view only inpatient care at a hospital or outpatient care elsewhere. A user may examine specific pins to view more detailed information. For example, window 442 shows that the particular patient visited Dr. Smith's rheumatology clinic on Aug. 14, 2013 as well as a link to a patient note prepared by Dr. Smith. It will be appreciated that other immunosuppression features may be included in patient timeline 400 in other embodiments. For example, a timeline documenting a patient's surgical procedures may also be included.

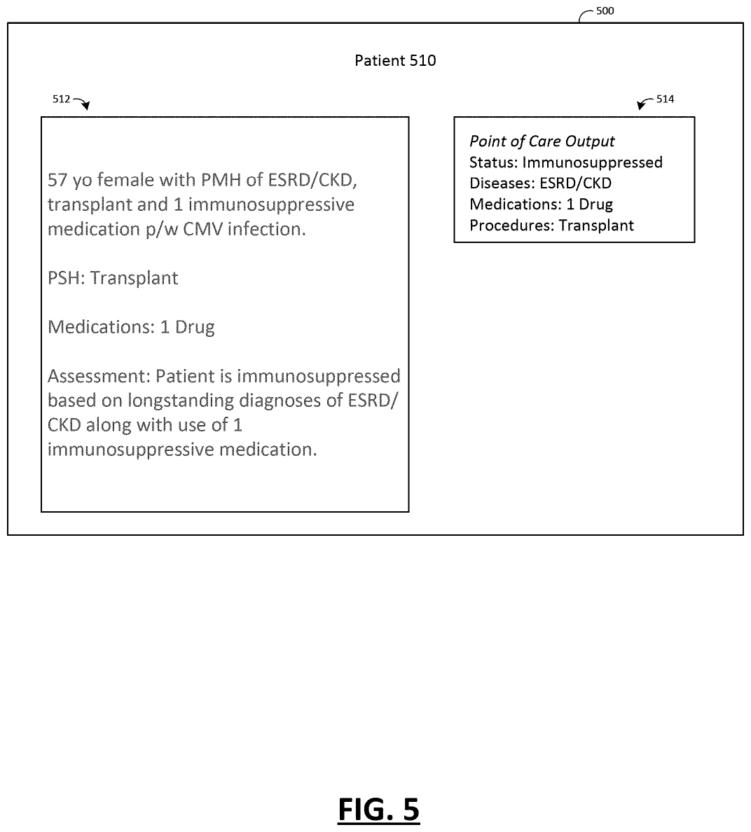

FIG. 5 illustrates an example individualized output displayed by the immunocompromised prediction system 120. Individualized output 500 comprises information regarding the specific patient 510. For example, output 500 includes windows 512 and 514. Window 512 lists relevant immunosuppression features for patient 510, such as the patient's age, sex, known diseases, opportunistic infections, transplant and medication histories, and an assessment of whether patient 510 is immunosuppressed. Window 514 summarizes the data and assessment presented in window 512 and displays it to the user such that a clinician may quickly digest the information and provide proper treatment, advice, or medications and adjust any medical protocols in view of the patient's immunosuppressed status. For example, because immunosuppressed patients have a higher risk for community-acquired infections, a health professional may take extra precautions during the hospital discharge process, such as by specifically informing the patient of his immunocompromised status and providing recommendations to reduce the risk of infection during daily life outside of the hospital. Additionally, immunosuppressed patients may be considered for certain clinical trials but excluded in others. For example, a doctor may ordinarily recommend a clinical trial of cancer immunotherapy but may avoid recommending such a trial patient 510 in order to maximize the probability of an accurate treatment response. While the information in output 500 may be used by a health professional, it will be appreciated that such information may also be displayed to the patient or other authorized user in some embodiments. For example, patient 510 may discover that he is immunosuppressed and therefore uniquely eligible to receive certain vaccinations.

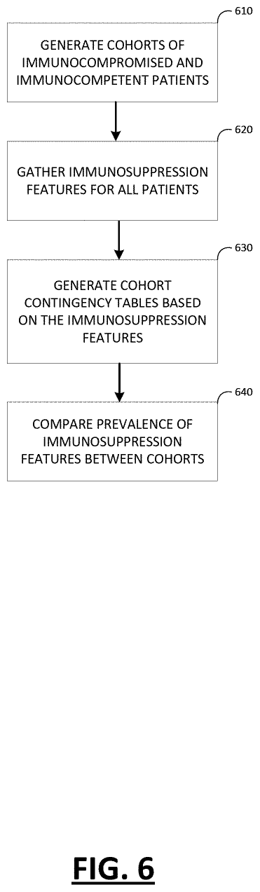

FIG. 6 illustrates an example flow diagram for preparing and validating data for the immunocompromised prediction system. In block 610, the system generates cohorts of immunocompromised and immunocompetent patients. In some embodiments, the prediction system may generate an immunocompromised cohort of patients where each patient in the cohort is associated with clinician records that mention immunosuppression. In some embodiments, the system may group patients along additional factors, such as by the patient's age or sex, and each cohort may be associated with a target index date, such as the first date on which a clinical note indicated that a particular patient was immunosuppressed. While the prediction system may sort patients into cohorts after evaluating patient information, in some embodiments the prediction system may be supplied with data that is already curated. For example, a user may provide the prediction system with a control group of patients with known immunosuppression features and known a predefined immunosuppression status for purposes of training.

In block 620, the prediction system may gather immunosuppression features for all patients among the cohorts. In some embodiments, the features gathered by the immunocompromised prediction system may be obtained from structured data commonly utilized in electronic medical records systems. For example, data generation module 122 may obtain structured data referring to disease codes (e.g., ICD-10, ICD-9, SNOMED, etc.), procedure codes (e.g., CPT), and medication records (e.g., documentation of medications ordered or administered to patients). In some embodiments, the prediction system may extract a list of specific features associated with the particular patient. For example, the system may obtain immunodeficiency diagnosis codes, cancer diagnosis codes, opportunistic infection codes, autoimmune disease diagnosis codes, chronic disease diagnosis codes, a list of immunosuppressive medications ordered and administered, and transplant procedure codes for each patient. In some embodiments, the prediction system may generate immunodeficiency features from unstructured clinical text using pre-trained natural language processing models. For example, the prediction system may use a disease-diagnosis model, such as a BERT-based, named-entity recognition and sentiment association model, to identify diagnoses from clinical notes. In some embodiments, the model may be optimized to identify a range of criteria or identify a specific feature. For example, in some embodiments the model may identify specific words such as “immunosuppression” in clinical notes.

The prediction system generates cohort contingency tables in block 630. In some embodiments, the cohort tables are based at least partly on immunosuppression features collected in block 620. For example, the cohort contingency table may contain rows depicting each immunosuppression feature collected from block 620 and columns for each year before a predetermined index date. In some embodiments, the index date associated with the immunosuppressed cohort may be the date a patient was first discovered to be immunosuppressed. For the immunocompetent cohort, the index date may be defined as the index date of a corresponding immunosuppressed patient. It will be appreciated that the prediction system is not limited to using only contingency tables. The prediction system may represent or format data in any other format (e.g., relational database, array, linked list, etc.).

In block 640, the prediction system compares the prevalence of immunosuppression features between cohorts. In some embodiments, the system may compute an odds ratio or compute a Fisher p-value for each immunosuppression feature. In some embodiments, the system may tally the number of immunosuppression features present in a time interval relative to the defined index date. In some embodiments, the system may conduct an analysis (e.g., an ROC analysis) to determine the sensitivity and specificity for immunosuppression classification. Additionally, the system may perform a logistic regression analysis to evaluate how strong the association for each immunosuppression feature with a known immunosuppression status. In doing so, the system may calculate the values for specific regression coefficients associated with each of the immunosuppression features to ultimately calculate the odds of a patient belonging to an immunosuppressed classification. For example, the following equation may be used to calculate the odds of being classified as immunosuppressed based on a list of immunosuppression features, such as immunodeficiency, presence of solid cancer, metastic cancer, autoimmune diseases, chronic diseases, opportunistic infection, transplants, immunosuppressive medications, or other features:

Log Odds(Immunosuppressed)=β₀+β₁(Immunodeficiency)+β₂(Solid cancer)+β₃(Metastatic cancer)+β₄(Heme cancer)+β₅(Autoimmune disease)+β₆(Chronic disease)+β₇(Opportunistic infection)+β₈(Transplant)+β₉(Immunosuppressive meds)

wherein β₀ through β₉ are calculated regression coefficients. It will be appreciated that other embodiments may utilize an alternate combination of immunosuppression features. In some embodiments, the prediction system may generate weighting schemes for selected variables to indicate which immunosuppression features may impact the prediction more heavily. For example, if the prediction system learns, through a machine learning model, that the presence of an autoimmune disease is strongly correlated with an immunocompromised status, then the prediction system may assign a higher weight to this feature (reflected as a larger value of β₅) in the above equation in comparison to other coefficients. In some embodiments, the prediction system may also improve the accuracy of the immunosuppression classification by adjusting the list of selected immunosuppression features itself. For example, the prediction system may remove an immunosuppression feature such as “opportunistic infection” from consideration if the system iterates through multiple patients and finds no strong correlation between the “opportunistic infection” feature and the immunosuppression classification.

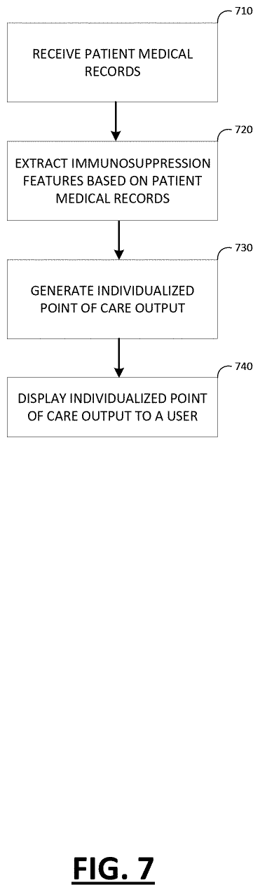

FIG. 7 illustrates an example flow diagram to return and display an individualized point of care output. In block 710, the system receives patient medical records, such as from electronic medical records systems used in hospitals or other health professional clinics. In block 720, the prediction system may extract immunosuppression features based on the received patient medical records. In some embodiments, the features collected and considered by the immunocompromised prediction system 120 may be obtained from structured data commonly utilized in electronic medical records systems. For example, data generation module 122 may obtain structured data referring to disease codes (e.g., ICD-10, ICD-9, SNOMED, etc.), procedure codes (e.g., CPT), and medication records (e.g., documentation of medications ordered or administered to patients). In some embodiments, data generation module 122 may extract specific information from each patient that may be relevant to predicting immunosuppression status. For example, data generation module 122 may obtain immunodeficiency diagnosis codes, cancer diagnosis codes, opportunistic infection codes, autoimmune disease diagnosis codes, chronic disease diagnosis codes, a list of immunosuppressive medications ordered and administered, and transplant procedure codes for each patient.

In block 730, the prediction system may generate an individualized point of care output for a specific patient. In some embodiments, the prediction system may generate the individualized point of care output based at least on previously validated data and assessments of immunosuppressed cohorts. In certain embodiments, previously validated data may be used to train a machine learning model to predict the likelihood that a particular patient belongs to an immunosuppression classification. For example, a control group may be initially supplied comprising a list of patients with known immunosuppression features (e.g., presence of autoimmune disease) and a predetermined immunosuppression status (e.g., immunosuppressed). After iterating through initial training data, the machine learning model in some embodiments may automatically associate certain immunosuppression features with a higher likelihood of obtaining an immunosuppressed status. For example, if the machine learning model receives as input a patient possessing the “chronic disease” immunosuppression feature, the machine learning model in some embodiments may automatically assign that immunosuppression feature a higher weighting and likelihood of immunosuppression in comparison to other features. In some embodiments, the trained immunosuppression machine learning model may output a numerical score indicating the likelihood that the patient is immunosuppressed. In some embodiments, the generated point of care output may comprise a list of patient information, such as the patient's age, history of disease and infection, transplants, and drug prescriptions, as well as an assessment of whether the patient is immunosuppressed. In block 740, the prediction system may output a visual display summarizing the generated individualized point of care output, such as the interactive patient timeline mentioned above in connection with FIG. 4 and the point of care output mentioned above in connection with FIG. 5. It will be appreciated that other methods and display configurations may be utilized to display a personalized immunosuppression assessment in other embodiments.

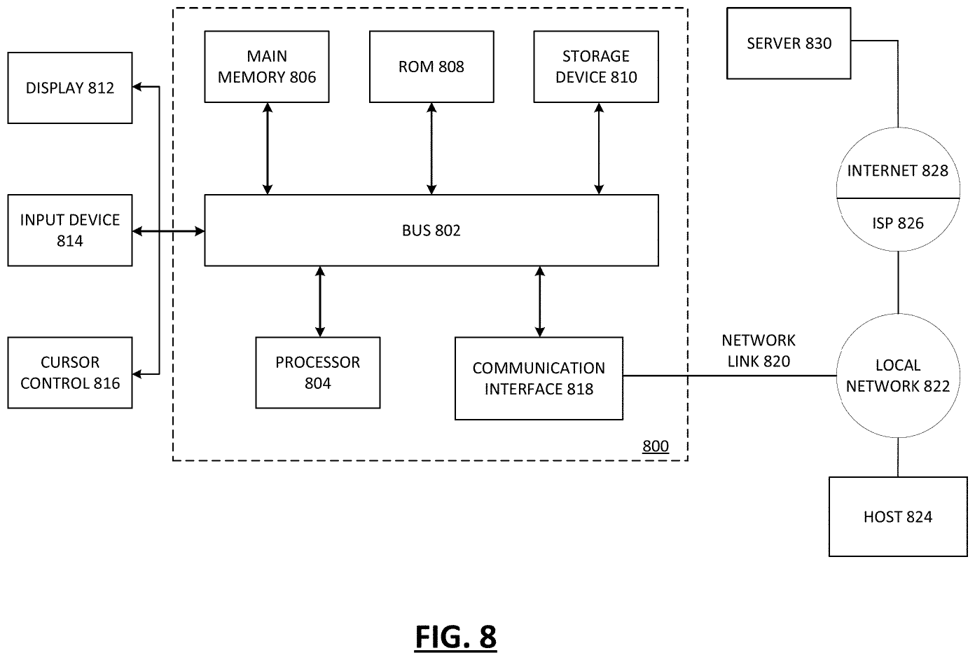

FIG. 8 is a block diagram that illustrates a computer system 800 upon which an embodiment of the invention may be implemented. Computer system 800 includes a bus 802 or other communication mechanism for communicating information, and a processor 804 coupled with bus 802 for processing information. Computer system 800 also includes a main memory 806, such as a random access memory (RAM) or other dynamic storage device, coupled to bus 802 for storing information and instructions to be executed by processor 804. Main memory 806 also may be used for storing temporary variables or other intermediate information during execution of instructions to be executed by processor 804. Computer system 800 further includes a read only memory (ROM) 808 or other static storage device coupled to bus 802 for storing static information and instructions for processor 804. A storage device 810, such as a magnetic disk or optical disk, is provided and coupled to bus 802 for storing information and instructions.

Computer system 800 may be coupled via bus 802 to a display 812, such as a cathode ray tube (CRT), for displaying information to a computer user. An input device 814, including alphanumeric and other keys, is coupled to bus 802 for communicating information and command selections to processor 804. Another type of user input device is cursor control 816, such as a mouse, a trackball, or cursor direction keys for communicating direction information and command selections to processor 804 and for controlling cursor movement on display 812. This input device typically has two degrees of freedom in two axes, a first axis (e.g., x) and a second axis (e.g., y), that allows the device to specify positions in a plane.

The invention is related to the use of computer system 800 for implementing the techniques described herein. According to one embodiment of the invention, those techniques are performed by computer system 800 in response to processor 804 executing one or more sequences of one or more instructions contained in main memory 806. Such instructions may be read into main memory 806 from another machine-readable medium, such as storage device 810. Execution of the sequences of instructions contained in main memory 806 causes processor 804 to perform the process steps described herein. In alternative embodiments, hard-wired circuitry may be used in place of or in combination with software instructions to implement the invention. Thus, embodiments of the invention are not limited to any specific combination of hardware circuitry and software.

The term “machine-readable medium” as used herein refers to any medium that participates in providing data that causes a machine to operate in a specific fashion. In an embodiment implemented using computer system 800, various machine-readable media are involved, for example, in providing instructions to processor 804 for execution. Such a medium may take many forms, including but not limited to, non-volatile media, volatile media, and transmission media. Non-volatile media includes, for example, optical or magnetic disks, such as storage device 810. Volatile media includes dynamic memory, such as main memory 806. Transmission media includes coaxial cables, copper wire and fiber optics, including the wires that comprise bus 802. Transmission media can also take the form of acoustic or light waves, such as those generated during radio-wave and infra-red data communications.

Common forms of machine-readable media include, for example, a floppy disk, a flexible disk, hard disk, magnetic tape, or any other magnetic medium, a CD-ROM, any other optical medium, punchcards, papertape, any other physical medium with patterns of holes, a RAM, a PROM, and EPROM, a FLASH-EPROM, any other memory chip or cartridge, a carrier wave as described hereinafter, or any other medium from which a computer can read.

Various forms of machine-readable media may be involved in carrying one or more sequences of one or more instructions to processor 804 for execution. For example, the instructions may initially be carried on a magnetic disk of a remote computer. The remote computer can load the instructions into its dynamic memory and send the instructions over a telephone line using a modem. A modem local to computer system 800 can receive the data on the telephone line and use an infra-red transmitter to convert the data to an infra-red signal. An infra-red detector can receive the data carried in the infra-red signal and appropriate circuitry can place the data on bus 802. Bus 802 carries the data to main memory 806, from which processor 804 retrieves and executes the instructions. The instructions received by main memory 806 may optionally be stored on storage device 810 either before or after execution by processor 804.

Computer system 800 also includes a communication interface 818 coupled to bus 802. Communication interface 818 provides a two-way data communication coupling to a network link 820 that is connected to a local network 822. For example, communication interface 818 may be an integrated services digital network (ISDN) card or a modem to provide a data communication connection to a corresponding type of telephone line. As another example, communication interface 818 may be a local area network (LAN) card to provide a data communication connection to a compatible LAN. Wireless links may also be implemented. In any such implementation, communication interface 818 sends and receives electrical, electromagnetic or optical signals that carry digital data streams representing various types of information.

Network link 820 typically provides data communication through one or more networks to other data devices. For example, network link 820 may provide a connection through local network 822 to a host computer 824 or to data equipment operated by an Internet Service Provider (ISP) 826. ISP 826 in turn provides data communication services through the world wide packet data communication network now commonly referred to as the “Internet” 828. Local network 822 and Internet 828 both use electrical, electromagnetic or optical signals that carry digital data streams. The signals through the various networks and the signals on network link 820 and through communication interface 818, which carry the digital data to and from computer system 800, are exemplary forms of carrier waves transporting the information.

Computer system 800 can send messages and receive data, including program code, through the network(s), network link 820 and communication interface 818. In the Internet example, a server 830 might transmit a requested code for an application program through Internet 828, ISP 826, local network 822 and communication interface 818.

The received code may be executed by processor 804 as it is received, and/or stored in storage device 810, or other non-volatile storage for later execution. In this manner, computer system 800 may obtain application code in the form of a carrier wave.

Moreover, some or all of the structure and functionality of the embodiments described above may be implemented on a single processor or a single server. Alternatively, some or all of the structure and functionality of the embodiments described above may be implemented via a distributed network of processors and servers located in the same or different remote locations.

As will be apparent to one of ordinary skill in the art from a reading of this disclosure, the disclosed subject matter can be embodied in forms other than those specifically disclosed above. The particular embodiments described above are, therefore, to be considered as illustrative and not restrictive. Those skilled in the art will recognize, or be able to ascertain, using no more than routine experimentation, numerous equivalents to the specific embodiments described herein. The scope of the invention is as set forth in the appended claims and equivalents thereof, rather than being limited to the examples contained in the foregoing description.

1. A method comprising:
receiving, by one or more computer processors, a set of medical records associated with a patient;
extracting, by the one or more computer processors, a set of immunosuppression features based on the set of medical records;
estimating, by the one or more computer processors, a likelihood of immunosuppression of the patient based on the set of immunosuppression features;
generating, by the one or more computer processors, an immunosuppression output comprising one or more features among the set of immunosuppression features and an immunosuppression classification based on the likelihood of immunosuppression of the patient; and
displaying, by the one or more computer processors, the immunosuppression output through at least one interface.

2. The method of claim 1, wherein the set of immunosuppression features includes at least one of an immunodeficiency diagnosis code, a cancer diagnosis code, an opportunistic infection code, an autoimmune disease diagnosis code, a chronic disease diagnosis code, a list of immunosuppressive medications administered, or a transplant procedure code.

3. The method of claim 1, wherein extracting the set of immunosuppression features comprises filtering the set of immunosuppression features to include features occurring within a predetermined time frame.

4. The method of claim 1, wherein extracting the set of immunosuppression features comprises using a natural language processing model to process unstructured text data within the set of patient records.

5. The method of claim 1, wherein estimating the likelihood of immunosuppression of the patient comprises evaluating a predictive power associated with at least one feature among the set of immunosuppression features.

6. The method of claim 5, wherein evaluating the predictive power associated with the at least one feature comprises:
receiving a plurality of sets of medical records associated with a plurality of patients;
identifying an immunosuppressed cohort and an immunocompetent cohort among the plurality of patients based on the plurality of sets of medical records;
extracting the at least one feature from the plurality of sets of medical records and identifying, for the immunosuppressed cohort, a number of times the at last one feature is present within a predetermined time interval before a date that the immunosuppressed patients in the immunosuppressed cohort were determined to be immunosuppressed; and
comparing a prevalence of the at least one feature in the time interval between the immunosuppressed cohort and the immunocompetent cohort.

7. The method of claim 6, wherein evaluating the predictive power associated with the at least one feature further comprises calculating a probability that the patient is immunosuppressed based on a determination that the at least one feature is present in the set of medical records.

8. The method of claim 1, wherein displaying the immunosuppression output comprises displaying an interactive patient timeline that represents of the set of immunosuppression features as a function of time.

9. The method of claim 8, wherein the interactive patient timeline allows a user to filter the set of immunosuppression features that are displayed.

10. A system comprising:
a non-transitory memory; and
one or more computer processors configured to read instructions from the non-transitory memory that, when executed, cause the one or more computer processors to perform operations comprising:
receiving a set of medical records associated with a patient;
extracting a set of immunosuppression features based on the set of medical records;
estimating a likelihood of immunosuppression of the patient based on the set of immunosuppression features;
generating an immunosuppression output comprising one or more features among the set of immunosuppression features and an immunosuppression classification based on the likelihood of immunosuppression of the patient; and
displaying the immunosuppression output through at least one interface.

11. The system of claim 10, wherein the set of immunosuppression features includes at least one of an immunodeficiency diagnosis code, a cancer diagnosis code, an opportunistic infection code, an autoimmune disease diagnosis code, a chronic disease diagnosis code, a list of immunosuppressive medications administered, or a transplant procedure code.

12. The system of claim 10, wherein extracting the set of immunosuppression features comprises filtering the set of immunosuppression features to include features occurring within a predetermined time frame.

13. The system of claim 10, wherein extracting the set of immunosuppression features comprises using a natural language processing model to process unstructured text data within the set of patient records.

14. The system of claim 10, wherein estimating the likelihood of immunosuppression of the patient comprises evaluating a predictive power associated with at least one feature among the set of immunosuppression features.

15. The system of claim 14, wherein evaluating the predictive power associated with the at least one feature comprises:
receiving a plurality of sets of medical records associated with a plurality of patients;
identifying an immunosuppressed cohort and an immunocompetent cohort among the plurality of patients based on the plurality of sets of medical records;
extracting the at least one feature from the plurality of sets of medical records and identifying, for the immunosuppressed cohort, a number of times the at least one feature is present within a predetermined time interval before a date that the immunosuppressed patients in the immunosuppressed cohort were determined to be immunosuppressed; and
comparing a prevalence of the at least one feature in the time interval between the immunosuppressed cohort and the immunocompetent cohort.

16. The system of claim 15, wherein evaluating the predictive power associated with the at least one feature further comprises calculating a probability that the patient is immunosuppressed based on a determination that the at least one feature is present in the set of medical records.

17. The system of claim 10, wherein displaying the immunosuppression output comprises displaying an interactive patient timeline that represents of the set of immunosuppression features as a function of time.

18. The system of claim 17, wherein the interactive patient timeline allows a user to filter the set of immunosuppression features that are displayed.

19. A non-transitory computer-readable medium storing instructions that, when executed by one or more computer processors, cause the one or more computer processors to perform operations comprising:
receiving a set of medical records associated with a patient;
extracting a set of immunosuppression features based on the set of medical records;
estimating a likelihood of immunosuppression of the patient based on the set of immunosuppression features;
generating an immunosuppression output comprising one or more features among the set of immunosuppression features and an immunosuppression classification based on the likelihood of immunosuppression of the patient; and
displaying the immunosuppression output through at least one interface.

20. The non-transitory computer-readable medium of claim 19, wherein the set of immunosuppression features includes at least one of an immunodeficiency diagnosis code, a cancer diagnosis code, an opportunistic infection code, an autoimmune disease diagnosis code, a chronic disease diagnosis code, a list of immunosuppressive medications administered, or a transplant procedure code.

21. The non-transitory computer-readable medium of claim 19, wherein extracting the set of immunosuppression features comprises filtering the set of immunosuppression features to include features occurring within a predetermined time frame.

22. The non-transitory computer-readable medium of claim 19, wherein extracting the set of immunosuppression features comprises using a natural language processing model to process unstructured text data within the set of patient records.

23. The non-transitory computer-readable medium of claim 19, wherein estimating the likelihood of immunosuppression of the patient comprises evaluating a predictive power associated with at least one feature among the set of immunosuppression features.

24. The non-transitory computer-readable medium of claim 23, wherein evaluating the predictive power associated with the at least one feature comprises:
receiving a plurality of sets of medical records associated with a plurality of patients;
identifying an immunosuppressed cohort and an immunocompetent cohort among the plurality of patients based on the plurality of sets of medical records;
extracting the at least one feature from the plurality of sets of medical records and identifying, for the immunosuppressed cohort, a number of times the at least one feature is present within a predetermined time interval before a date that the immunosuppressed patients in the immunosuppressed cohort were determined to be immunosuppressed; and
comparing a prevalence of the at least one feature in the time interval between the immunosuppressed cohort and the immunocompetent cohort.

25. The non-transitory computer-readable medium of claim 24, wherein evaluating the predictive power associated with the at least one feature further comprises calculating a probability that the patient is immunosuppressed based on a determination that the at least one feature is present in the set of medical records.

26. The non-transitory computer-readable medium of claim 19, wherein displaying the immunosuppression output comprises displaying an interactive patient timeline that represents of the set of immunosuppression features as a function of time.

27. The non-transitory computer-readable medium of claim 26, wherein the interactive patient timeline allows a user to filter the set of immunosuppression features that are displayed.
